(19) United States
(12) Patent Application Publication
(10) Pub. No.: US 2007/0178497 A1
(43) Pub. Date: Aug. 2, 2007

(54) Identification

(76) Inventors: Peter David Gill, Birmingham (GB); Javaid Iqbal Hussain, Birmingham (GB); Adam Spencer Long, Birmingham (GB); Gillian Tully, London (GB)

Correspondence Address: MERCHANT & GOULD PC, P.O. BOX 2903, MINNEAPOLIS, MN, 55402-0903, US

(21) Appl. No.: 11/615,046

(22) Filed: Dec. 22, 2006

Related U.S. Application Data
(63) Continuation of application No. 10/369,193, filed on Feb. 14, 2003, now abandoned.
Application No. 10/369,193 is a continuation of application No. 09/745,687, filed on Dec. 22, 2000, now abandoned.

(30) Foreign Application Priority Data
Dec. 22, 1999 (GB) 9930307.5

(51) Int. Cl. C12Q 1/68; G06F 19/00

(52) U.S. Cl. 435/6; 702/20

(57) ABSTRACT

The invention provides a method for obtaining additional information about DNA mixtures arising from a variety of sources and/or a variety of concentrations. In particular, the invention provides a method for indicating the likelihood that a DNA mixture arose from sources of a defined type where: the DNA mixture is formed by DNA samples from more than one source, the method involving the determination of the identity of the alleles present at a locus for the DNA in the mixture; determining a first probability function for the situation where the DNA mixture is formed from samples arising from the given person and from a first other person; determining a second probability function for the situation where the DNA mixture is formed from samples arising from a second other person and a first other person; using the first probability function as numerator and the second probability function as denominator in determining a likelihood ratio for the mixture having arisen from the defined type of sources considered in the first probability function; determining such likelihood ratios for a plurality of loci; and combining the likelihood ratios to give a combined likelihood ratio for the mixture having arisen from the defined type of sources considered in the first probability function.

This invention concerns improvements in and relation to identification, particularly in the field of forensic science, and particular but not exclusively relating to identification techniques based on the use of single nucleotide polymorphism.

In a wide variety of situations it is desirable to be able to obtain information about the contributors to a mixture of DNA. Existing techniques are either limited in terms of the range of known factors which must be available for meaningful results to be obtained and/or limited in terms of the concentration of DNA which must be available from each of the sources to be considered and/or the relative proportions of the DNA sources contributing to the mixture.

The present invention aims to provide a technique which is more versatile in terms of the type of situation which can meaningfully be considered and/or be more useful in terms of the range of concentration which can be usefully considered and/or be more useful in terms of the proportions of the DNA contributed to a mixture by more than one contributor which can be considered.

According to a first aspect of the present invention we provide a method for indicating the likelihood that a DNA mixture arose from sources of a defined type where the DNA mixture is formed by DNA samples from more than one source, the method involving:

the determination of the identity of the alleles present at a locus for the DNA in the mixture;

determining a fist probability function for the situation where the DNA mixture is formed from samples arising from the given person and from a first other person;
determining a second probability function for the situation where the DNA mixture is formed from samples arising from a second other person and a first other person;

using the first probability function as numerator and the second probability function as denominator in determining a likelihood ratio for the mixture having arisen from the defined type of sources considered in the first probability function;

determining such likelihood ratios for a plurality of loci; and

combining the likelihood ratios to give a combined likelihood ratio for the mixture having arisen from the defined type of sources considered in the first probability function.

The defined type may assign an origin to one or both of the sources contributing to the mixture. The given person may be a suspect or other known person under investigation.

The first other person, particularly where the mixture is being considered as potentially arising from a suspect and an unknown person, may be a known person. The second other person in such cases may also be an unknown person.

The first other person, particularly where the mixture is being considered as potentially arising from a suspect and a victim, may be a known person, such as the victim. The second other person in such cases may be an unknown person, particularly neither the suspect or victim.

Preferably the mixture arises from only two sources.

The identity of the alleles may be determined using techniques for identifying single nucleotide polymorphisms.

Preferably the first probability function is the probability that the defined type provides one or both of the mixture sources, ideally based on the frequency of occurrence of the possible allele combinations which could generate the identified allele identity or identities for that locus. The identity of the alleles at a locus, from the two sources, may be the same or different

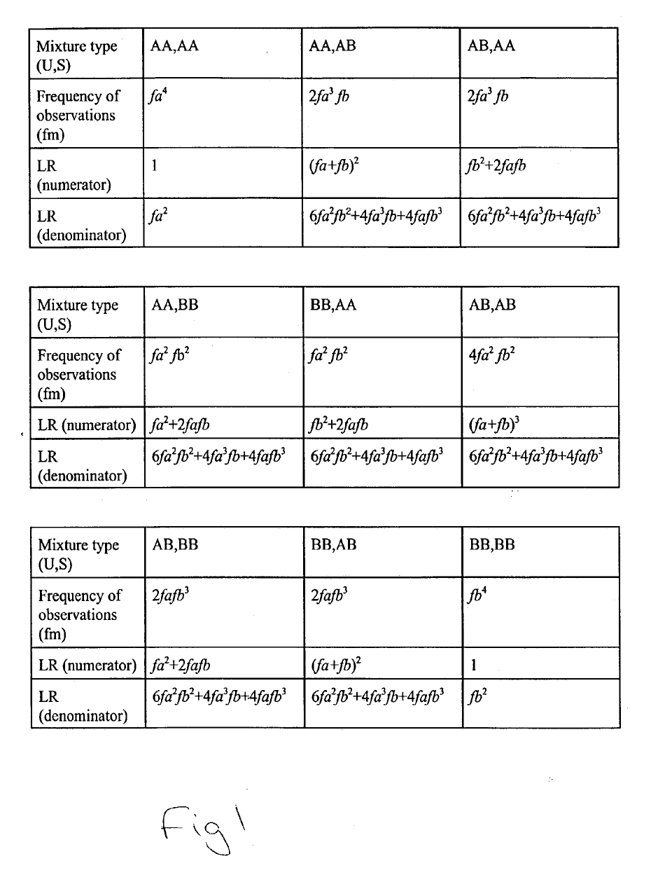

In a first embodiment of the method, where the defined type is the given person and an unknown person,, the first function may be based on the frequency of occurrence of the different possible allele combinations for the unknown person which are possible knowing the given persons alleles at that locus. The first function, preferably the numerator thereof, may be any one or more of the numerator functions set out in FIG. 1. The first function, preferably the numerator thereof, may be the numerator function set out in FIG. 1 for the given respective allele identity set out in FIG. 1.

In a second embodiment of the method, where the defined type is the given person and the first other person is a known person, the first function may be defined as 1.

Preferably the second probability function is the probability that the first and second other persons provide the identity for the mixture sources, ideally based on the frequency of occurrence of possible allele combinations which could have generated the identified allele identity of identities for that locus. The identity of the alleles at a locus, from the two sources, may be the same or different.

In a first embodiment of the method, where the defined type is the given person and an unknown person, the second function may be based on the frequency of occurrence of the different allele combinations which are possible from the two unknown persons which give the allele identity or identities obtained. The second function, preferably the denominator thereof, may be any one or more of the denominator functions set out in FIG. 1. The second function, preferably the denominator thereof, may be the denominator function set out in FIG. 1 for the given respective allele identity set out in FIG. 1.

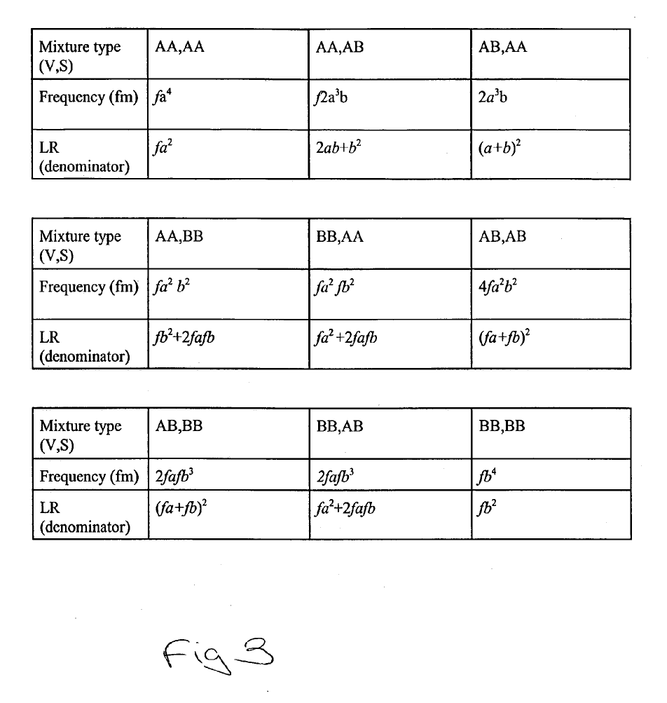

In a second embodiment of the method, where the defined type is the given person and the first other person is a known person, the second function may be based on the frequency of occurrence of the different possible allele combinations for the unknown person which are possible knowing the known person's alleles at that locus. The second function, preferably the denominator thereof, may be any one or more of the denominator functions set out in FIG. 3. The second function, preferably the denominator thereof, may be the denominator function set out in FIG. 3 for the given respective allele identities set out in FIG. 3.

The method may be applied to two or more loci, but is preferably applied to at least 20 loci and still more preferably at least 30 loci. The method may be applied to 50 or more, 100 or more, 150 or more or even 200 or more loci to increase the statistical significance of the results.

The combined likelihood ratio may be obtained by multiplying the individual likelihood ratios together.

To estimate the optimum number of loci used, preferably in an array, a theoretical likelihood ratio way be used, ideally calcalated from:

$$\overline{{LR}_{n}} = {\prod }_{m = 1}^{mp} {LR}^{( fm \times n )}$$

where n is the number of loci; mp is the number of possible allele identities for a simple mixture; LR is the likelihood ratio; LR is the combined likelihood ratio; and fm is the proportion of an array of a loci having a particular mixture type m.

The proportions of the loci (fm) having the specified identities (mixture type) may be as set out below:

Mixture type (U, S)

| | AAAA | AAAB | ABAA |
|---|---|---|---|
| Frequency of | fα⁴ | 2fα³fb | 2fα³fb |
| observations (fα) | | | |

Mixture type (U, S)

| | AABB | BBAA | ABAB |
|---|---|---|---|
| Frequency of | fα²fb² | fα²fb² | 4fα²fb² |
| observations (fα) | | | |

Mixture type (U, S)

| | ABBB | BBAB | BBBB |
|---|---|---|---|
| Frequency of | 2fαfb³ | 2fαfb³ | fb⁴ |
| observations (fα) | | | |

Where the allele identity or identities of a given person and/or known first other person are under consideration, the method may include the determination of the allele identity or identities at one or more of the loci under consideration from DNA obtained only from the given person or known first person.

In an embodiment of the invention, particularly where the defined type is the given person and the first other person is a known person, such as a victim, it is preferred that at least some of the loci considered in the method are those in which the given person and first other person are known to differ in allele identity. The method may consider only loci at which the given person and known first person have alleles which are different.

In an embodiment of the invention, particularly where the defined type is the given person and the first other person is a known person, such as a victim, the method may consider loci at which the given person and known first person are known to have the same homozygous allele identity. Preferably in such cases the method includes the establishment of a probability value that the other identity, for instance AA or BB, is absent. The probability value may involve an investigation of the background noise level from the allele identity investigating process, for instance a PCR based amplification process. The investigation may involve the introduction of one or more negative control samples. The investigation may involve the determination of a cumulative probability density function for one or more or all of the negative controls. This function may be used to establish the level and/or proportion of DNA in the mixture which would have given detection of the identity being established as absent. The level and/or proportion may be compared with other information thereon.

In an embodiment of the invention, particularly where the defined type is the given person and the first other person is a known person, such as a victim, the method may involve the establishment of a probability value that the given person's allele identity or identities has not been detected. In one instance, the probability value may relate to the given person's allele identity being different from that of the known first other person's. The known first other person's allele identity may be AA or BB, where A and B designate the two possible allele identities at that SNP. In such cases, the given person's allele identities accounted for may be BB, BA, AB where the first other person's identity is AA and/or the given person's allele identities accounted for may be AA, BA, AB where the first other person's identity is BB. The probability value may be accounted for by the equation:

$$LR = \frac{p ( B \neq null )}{[ 2 ab + {b}^{2} ] p ( B \neq null ) + {a}^{2} p ( B = null )}$$

where a and b are allele frequencies of A and B respectively.

In a second instance, the probability value may relate to the given person's allele identity being the same as that of the known first other person's. The given person and the known first other person's allele identity may be AA or BB, where A and B designate the two possible allele identities at that SNP. In such cases, the possibility that the mixture was formed by a sample from a second other person, rather an the given person, may be, accounted for. In such cases, the second other person's allele identities accounted for may be BB, BA, AB where the given person and first other person's identity is AA and/or the second other person's allele identities accounted for may be AA, BA, AB where the given person and the first other person's identity is BB. The probability value may be accounted for by the equation:

$$LR = \frac{p ( B = null )}{[ 2 ab + {b}^{2} ] p ( B \neq null ) + {a}^{2} p ( B = null )}$$

where a and b are allele frequencies of A and B respectively.

The method may further include the prediction of the proportion of the mixture arising from the person other than the first other person, for instance from the suspect as the given person. The method may include an estimate or calculation of a value for p(null). The value for p(null) may be calculated from a cumulative probability density function. The calculation may be derived from experimental data obtained by probing negative controls with respect to one or more allele identities.

Various embodiment of the invention will now be described, by way of example only, and with reference to the accompanying drawings in which:

FIG. 1 illustrates in tabular form the likelihood ratio numerator and denominator for each of the nine possible phenotype combinations when a mixture is under consideration as coming from an unknown individual and a suspect, with the proportion of mixture phenotypes expected (fm) also provided;

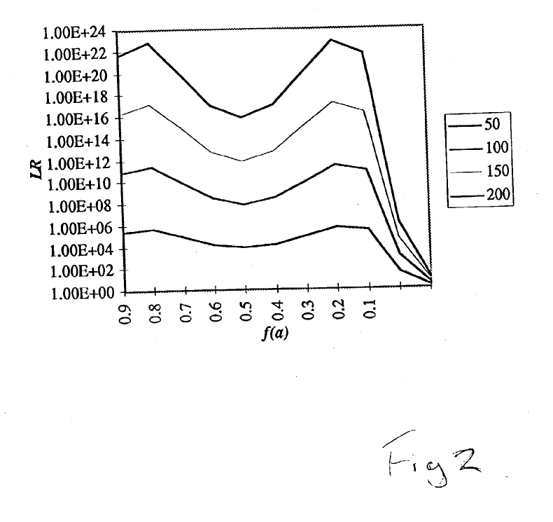

FIG. 2 is a plot of combined likelihood ratios for arrays ranging from 50 to 200 loci when there is a mixture under consideration as coming from a suspect and an unknown individual;

FIG. 3 illustrates in tabular form the likelihood ratio denominator for each of the nine possible phenotype combinations when a mixture is under consideration as coming from a victim and a suspect;

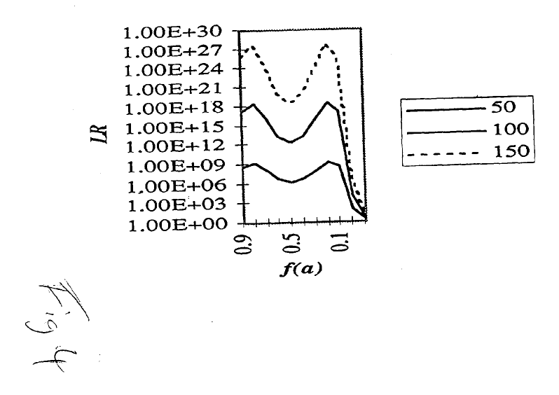

FIG. 4 is a plot of combined likelihood ratios for arrays ranging from 50 to 150 loci where there is a mixture under consideration as coming from a suspect and a victim;

FIG. 5 illustrates in tabular form DNA profile results for the victim and suspect in the first two columns with the multiplex test being specifically directed towards the alleles that are not found in the victim, as shown in the difference column with results column showing those expected if the suspect is the perpetrator (there are at least two possible genotypes to consider in the likelihood ratio where frequencies have then been incorporated into the calculation);

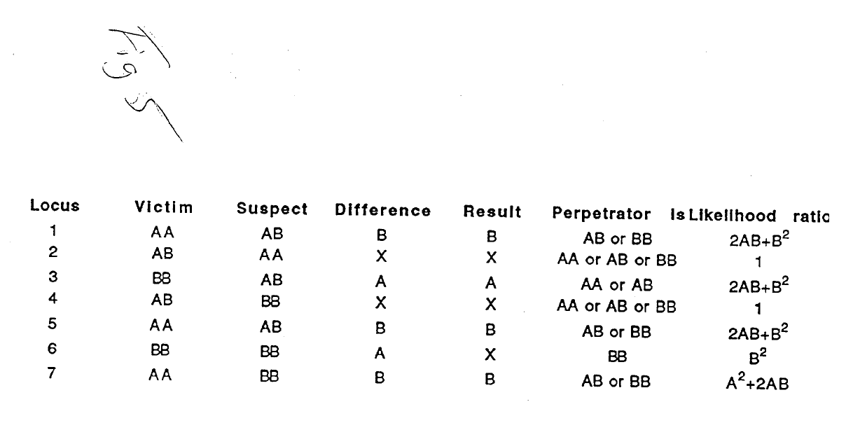

FIG. 6 exemplifies the results from four mitochondrial loci multiplex together using the universal primer approach, with sample 1 designated 247G; 195T; 152T; 146T; sample 2 designated 247G; 195C; 152C; 146C; and sample 3 is designed 247G; 195T: 152C; 146C;

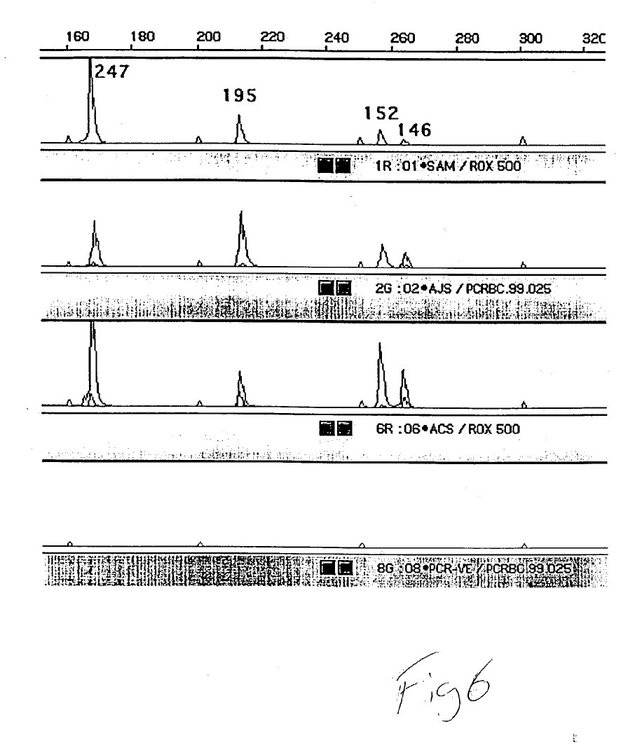

FIG. 7 illustrates a demonstration of amplification specificity and sensitivity of detection of mitochondrial DNA with the left hand series of figures showing detection of the major component of the mixture coding for the mt0073G polymorphism and the right hand series showing detection of the minor component of the mixture, coding for the mt0073A polymorphism, established in a the sensitivity of the test at Circa 12.5 pg genomic DNA;

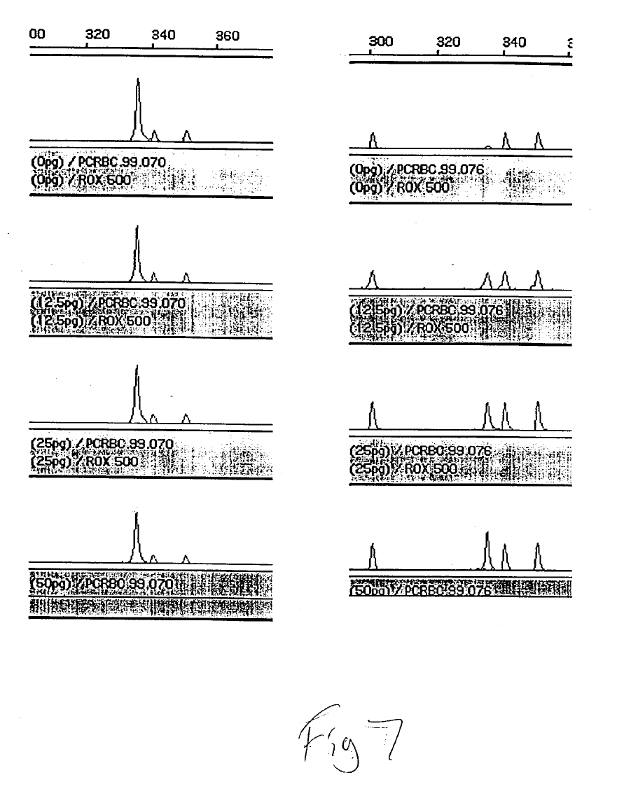

FIG. 8 illustrates results where primer 416A is tested against Gc1S-1S; 1f-1f; 2-2 and negative control;

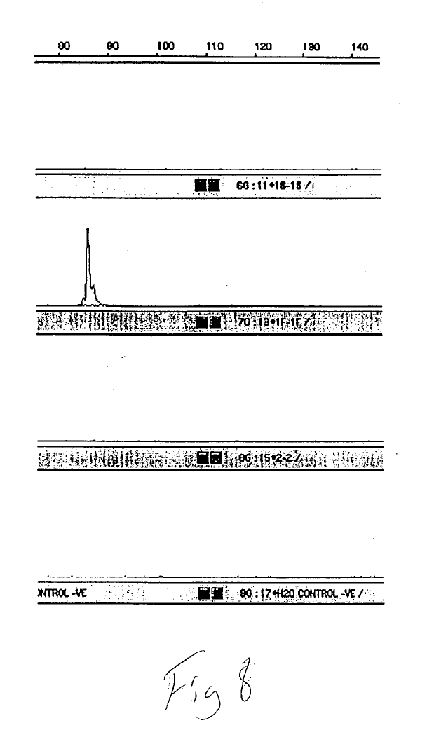

FIG. 9 illustrates singleplex reactions to test specifity of the forward primer 420G, which detects both the Gc1 polymorphisms, with the primer tested against a series of individuals—Gc1S-1S; 1f-1f; 2-2; and a negative control;

FIG. 10 illustrates results from a singleplex reaction to test specifity of forward primer 420T which detects Gc2 with the samples being from 1S-1S; 1f-1f;, 2; and negative control; and

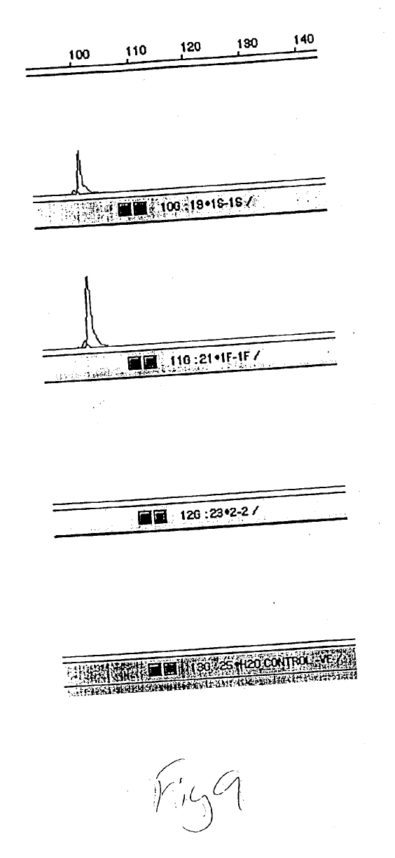

FIG. 11 demonstrates the limits of detection using a dilution series of a 1S-1S individual with samples prepared at 1 ng, 200 pg, 400 pg and 800 pg respectively.

### ANALYSIS OF MULTI-SOURCE SAMPLES

For any given single nucleotide polymorphism lotus there can only be two different alleles, In the following discussion these will be designated A and B.

Assuming that a DNA sample under consideration is a mixture with two contributors, if analysis of the mixture reveals just one allele appearing at the locus then both the contributors to the mixture must be homozygous for the same allele (AA; AA or BB; BB depending on the one allele determined).

If two alleles are visible in the experimental results then a considerable number of possibilities for the genotype combinations apply. Where two contributors are involved, the possible combinations are: AA, AB; AA, BB; AB, BB; AB, AB; and all of die reverse possibilities too. In total, nine possible genotype combinations exist for a two contributor mixture when both alleles are detected for a given locus.

This basic situation can be extended to relate to a two source mixture involving a suspect and an unknown individual and to a situation involving a two source mixture where a victim and a suspect are under consideration. Such situations where one of the contributors constitutes only a relatively minor part of the mixture can also be investigated using the technique set out in more detail below.

Contributors to the Mixture are Suspect and an Unknown Individual

For example, suppose that a blood stain is retrieved from a crime scene and the phenotypes are consistent with the combination of a suspect and an unknown individual. Two possible situations exist for which a likelihood ratio can be considered, first situation “C” where the contributors were the suspect and the unknown individual and secondly situation “not C” where the contributors are two uwkown individuals.

For any given locus under consideration, the calculation of the likelihood ration will depend upon the phenotype of the suspect and the alleles actually observed in the mixture. Three broad categories exist in this regard:

Category 1—where the suspect is homozygous (AA) and the profile shows just one allele and as a consequence the unknown must be AA also, thereby giving the LR=1/fa².

Category 2—where the suspect is homozygous (AA) and the mixture is AB and as a consequence the unknown must either by AB or BA or BB. In this case the probability of situation C=fb²+2fa fb and the likelihood of situation not C=6fa²fb²4fa³ fb+4fafb³ thereby giving a likelihood ratio=(2fafb+fb²)/(6fa²fb²+4fa³fb+4fafb³).

For Category 3—the suspect is heterozygous (AB) and the profile is AB and as a consequence the unknown must be AA, AB, BA or BB. In this case the probability of situation C=(fa+fb)² and the probability of situation not C is the same as for the Category 2 case thereby giving a likelihood ratio=(fa+fb)²/(6fa²fb²4fa³fb+4fafb³).

A complete list of the numerators and denominators for the likelihood ratios for the nine possible genotype combinations (m=1 to 9) are set out in the table of FIG. 1.

If an array of n different loci are considered, the proportion of an array of n loci having a particular mixture type is fm; and if for each locus there are mp=9 possible mixture phenotype combinations the combined likelihood ratio for the n loci is:

$$\overline{{LR}_{n}} = {\prod }_{m = 1}^{mp} {LR}^{( fm \times n )}$$

As an illustration of how the likelihood ratio plots vary for arrays involving 50 to 200 different loci (in the case of a mixture with a suspect and an unknown individual) reference is made to the plots set out in FIG. 2. In this illustration arrays of 50, 100, 150 and 200 loci were considered. The plot refers to the combined likelihood ratio for the n loci with the simplifying assumption that the allele frequency, f(a), for each locus is the same across all the loci. The combined likelihood ratio maximises when the frequency of allele a is high (0.8) or low (0.2). A battery of 50 loci with frequencies of alleles ranging between 0.1 and 0.9 will give a minimum LR of 10⁴ and a battery of 200 loci will give a minimum LR of 10¹⁶, thereby indicating significant statistical power.

Contributions to the Mixture are Victim and Suspect

In many situations, such as a typical rape forensic investigation, the mixture comprises contributions from both the victim and a suspect, thus there are two potential situations to be considered, situation “C” where the contributors to the mixture are the suspect and the victim, and situation “not C” where the contributors are the victim are an unknown individual.

Once again considering a mixture profile, with two alleles indicate a number of potential positions arise.

Firstly, if the profile comprises two alleles (AB) and the victim is known to be AB then the suspect may be AA, AB, BA or BB. The probability for situation C is thus=1. The probability for situation not C=(fa+fb)². This therefore gives a likelihood ratio=1/(fa+fb)².

Secondly, if the profile comprises two alleles (AB) and the victim is homozygous (AA) then the suspect is either AB, DA or BB. In this case the probability of situation C=1 once again, and the probability for situation not C=2fafb+fb². The likelihood ratio, therefore=1/(2fafb+fb²).

If he profile shows a single allele and both the victim and suspect are homozygote, (AA, AA), as a consequence the likelihood ratio=1/A².

The table of FIG. 3 illustrates the likelihood ratio denominator and frequency (fm) information for the potential nine genotype combinations. FIG. 4 illustrates in graphic form the plots obtained for 50, 100 and 150 loci based analysis in such cases.

Analysis of Mixtures with Minor Contribution from One Sample

As well as offering the above mentioned general consideration of the likelihood of the DNA in a sample from a number of sources applying, the present invention also offers the possibility of successful analysis even where one of the parties was only a minor contributor to the mixture, less than 10% of the mixture.

Techniques for analysing mixtures are known based around the use of a short tandem repeats (STR's) as described by Clayton et al. (1998) Analysis and Interpretation of Mixed Forensic Stains Using DNA STR Profiling. Int. J. Forensic Sci. 91, 55-70. The analysis of minor components and mixtures using STR based techniques, however, is particularly problematical when the minor component is present at a very low level (less than 1 in 10). Below this level, allele indications from the minor sample are close to the background noise and are difficult to distinguish as a result.

The technique of this invention can be applied to address such situations and still obtain meaningful results, however, particularly where the amplification set out in more detail below is applied. The technique of the present invention also offers different information from the analysis in the event of particular allele combinations.

In the technique, when a DNA sample is obtained which needs analysis and for which two contributors are suspected, then it is desirable to base the investigation of that sample on a method tailored to the DNA profile of the victim. When using SNP (single nucleotide polymorphism) based analysis, the most useful loci are those which are homozygous in the victim (where the victim is either AA or BB) as only then can detection of the other possible allele in the mixture imply information about the other contributor to the sample, possibly the perpetrator of a crime.

If the victim is heterozygous at the locus in question, then less useful information can be obtained since both alleles contributed by the victim will mask any alleles contributed by the perpetrator. Even so, where both the suspect and victim are the same homozygote (AA for instance) a new type of information can be provided. In such cases the allele B will be absent from the mixture and this can be confirmed using the present technique as the background level in the analysis is negligible, thus removing any argument that a BB contribution was present but was too small to detect.

FIG. 5 illustrates potential results for 7 different loci, including the victim allele identities, suspect allele identities, potential allele identities for a perpetrator and the likelihood ratio that the suspect was a contributor to the mixture under consideration. In this instance, therefore, only loci 1, 3, 5, 6 and 7 are potentially informative and worthy of analysis. This can be determined from an initial profile of the suspect's and victims DNA from a clean sample obtained from those persons.

Highly Specific Amplification Technique

Whilst the technique described in this application is applicable to all such analysis techniques, it offers particular advantages in providing information where the background noise from experimentally obtained data is minimised. In this regard reference is made to the technique described in the common applicants patent application number GB 9917307.2 filed 23 Jul. 1999 which describes a highly specific amplification technique which minimised background noise as a result. The contents of that application are fully incorporated herein by reference, particularly for the purposes of providing such a highly specific amplification technique.

Analysis of Mixtures with very Minor Contributions from One Sample

Even with the substantial reduction of background noise potential problems remain where one party's contribution is very much smaller than the other, less than 1 in 20 or even potentially down to situations where the contribution is less than 1 in 100. There are also potential problems where the mass of the mixture available for analysis is small (less than 25 pg). In both such cases there is a problem in that the alleles of the minor contributing party may not report to the result in a detectable way.

For STR's (short tandem repeats) even though the proportion of the mixture contributed by individual X relative to individual Y is similar between the different loci within the mixture, if the proportion of one party's contribution to the mixture is much lower than the other then the lower proportion allele is not necessarily observed in the results. This is a particular problem with STR's as the means of identification in that case depends on two pieces of information:- a), the mobility of allele in the electrophoretic gel and b) the relative concentration of intensity of the band which is used to assign the band to either the major or minor contributor of the mixture.

SNPs offer a considerable advantage in this area as the assay is purely quantitative, no mobility information need be obtained. The limits of detection, therefore are entirely dependent upon the levels of background noise inherent in the assay, as well as minimising the noise effect, the present invention offers the chance to provide further statistically relevant information by accounting for such potential non-reporting in its theory.

In the following explanation, cases where one parties contribution, a suspect for example, to the mixture is potentially very minor are considered and where, as a result, the allelic signal from that party's contribution is so close to the background noise threshold that it is difficult to distinguish from the noise.

Further assuming that the suspect=BB and the victim=AA then the profile should illustrate both A and B alleles. However, as previously stated if the proportion of the mixture contributed from suspect is very low or if the amount of DNA contributed by the suspect is very low then the B allele might not be detected in the results (potentially because it is swamped by the background noise of the system used). In such cases we need to interpret the information based on this potential non-observance (B is not null, it is present but not distinguished from the background noise) in relation to situation “C”, where the contributors are the suspect and the victim, and the situation “not C” where the contributors are the victim and an unknown person who is not the suspect

This can be expressed in the function set out below, where the numerator accounts for the alternative possible contributions from the suspect, as minor contributor. If B is present in reality, and not null, then the phenotypes which might contribute are AB, BA or BB; alternatively if B is not present even in reality (B is null) because the contributor does not possess this allele, this leaves AA as the only possibility. The function, the likelihood ratio, is summarised as follows:

$$LR = \frac{p ( B \neq null )}{[ 2 ab + {b}^{2} ] p ( B \neq null ) + {a}^{2} p ( B = null )}$$

Similarly, if the suspect is AA and the victim is AA, but the suspect contribution is very low, even if the profile only reveals A the possibility that the actual perpetrator is AB, BA or BB must be evaluated. This gives the function:

$$LR = \frac{p ( B = null )}{[ 2 ab + {b}^{2} ] p ( B \neq null ) + {a}^{2} p ( B = null )}$$

Given an estimate of the proportion of the minor contributor in the mixture, p(null) can be directly estimated from the cumulative probability distribution functions of the background controls for each locus. The lower the background signal is established to be, the lower p(null) value must be. Thus very greatly increased certainty can be expressed that the allele identity not reported in the results was due to it not being present in the samples which contribute to the mixture, rather than being there but not detected.

Experimental Illustrations of Invention

Tully et al (1996) described a mini-sequencing approach to analyse mitochondrial DNA SNPs. The SNPs listed in table 1 were analysed using the approach described above with universal G or universal C attached to the 5′ end of the primers listed. The sizes of each DNA fragment are known—when run on a gel, bands which are either JOE (green) or FAM (blue) labelled are visualised.

TABLE 1

| | | | |
|---|---|---|---|
| | | Sequence | 3′ polymorphism |
| | Primer sequence | Listing | used with |
| Position | used with Universal C | ID no. | Universal G |

Forward Primers

| | | | |
|---|---|---|---|
| 73 | GTATTTTCGTCTGGGGGGTA | 1 | G |
| 146 | GTCTGTCTTTGATTCCTGCCC | 2 | T |
| 152 | TTTGATTCCTGCCTCATCCC | 3 | T |
| 195 | ATATTACAGGCGAACATACC | 4 | T |
| 247 | GCTTGTAGGACATAATAATAACAATTA | 5 | G |

Reverse Primer

| | | |
|---|---|---|
| 326 | CAGAGATGTGTTTAAGTGCTGT | 6 |

NB. Universal primer C was dye labelled with FAM (blue) and Universal primer G was labelled with JOE (green).
Reaction Conditions
For each separate reaction:
DNTPs were at a final concentration of 35 mM
Perkin Elmer (PE) buffer was at a final concentration of 0.375mM with 0.375mM MgCl₂.
0.25 AmpliTaq (PE) was added to 50 ul reaction.
Primer concentrations are detailed separately with the examples given.
All phenotypes were verified by independent analysis using the mini-sequencing method of Tully et al (1996).

### Example 1: Multiplexed Mitochondrial DNA

Reaction Conditions:

DNTPs all at 10 mM;

Final concentration of 35 mM. PE buffer 15 mM MgCl2 per reaction MgCl2=0.375 mM. AmpliTaq=0.25 ul in 50 ul

In the following example, 1 uM of each of the forward primers and 2 uM of the reverse primer listed in table 1 was used in the reaction mixture. A 50 ul reaction containing 0.3 ng of genomic DNA was amplified through 8 cycles at 94C for 30sec; 57C for 30sec and 72C for 90sec. An aliquot of 5 ul of the reactant was then transferred into a second tube containing 1 uM of each forward universal primer and 1 uM of the reverse primer and 1 uM of the reverse universal primer. This was amplified for 22cycles at 94C for 30sec, 62C for 30sec and 72C for 90sec. Samples were electrophoresed on a ABD 377 automated sequencer with Rox 500 sizing standard. The negative control was treated under the same conditions, except that no DNA was added to the reaction.

Four mitochondrial loci were mutiplexed together using this universal primer approach. The results are illustrated in FIG. 6, where sample 1 is designated 247G; 195T; 152T; 146T; sample 2 is 247G; 195C; 152C146C; sample 3 is designated 247G; 195T; 152C; 146C. The regularly spaced small peak are size standards.

### Example 2

Elucidation of a Mixture where the Minor Component is<10 pg DNA (Genomic Equivalent)

In the next example, the results for which are illustrated in FIG. 7, mixtures were prepared with the major component coding for the mt0073G polymorphism (2 ng genomic DNA) and the minor component coding for the mt00326 polymorphism (0-50 pg). Amplified with forward primers, either mt0073-G or mt0073-A (1 uM) and the reverse primers mt00326 (1 uM) the cycling conditions were the same as described previously but the second round amplification was just 3 cycles.

In the first experiment, left hand side results, the primers used were mt0073-G (1 uM) and mt00326 (1 uM) whereas in the other experiment, right hand side results, the primers were, mt0073-A (1 uM) and mt00326 (1 uM). The results showed that even in the presence of very great excess of mt0073-G template, there was no mt0073-A background product detected. Similarly using just primer mt0073A there was no mt0073-G detected. The high specificity of the reaction demonstrated discrimination of minor components in mixtures down to extremely low levels of 12, 5 pg in a total—a mixture ratio of 1:200

### Example 3

Genomic DNA—Group Specific Component

The Gc single nucleotide polymorphisms have all been well characterised (Braun et al, 1992). In addition a large number of rare variants have been identified—the test described here only detect the common alleles

Gc 2, Gc1 F and Gc1S. Reynolds and Sensabaugh (1990) compared cDNA sequences of Yang et al (1985) and Cooke and David (1985). Although polymorphisms were observed at 4 different sites, the most informative are at codons 416 and 420, where single base changes result in an amino acid change. At triplet 416, GATA codes for an aspartic acid residue in the Gc2 and Gc1F phenotypes, whereas Gc1S has a glutamic acid residue determined by coin CAG. Amino acid 420 is a lysine residue in the Gc2 phenotype coded by AAG; a threonine residue in both Gc1 phenotypes is coded by ACG.

Four different forward primers were prepared to distinguish between the various polymorphisms (table 2, 3). These primers were attached at the 5′ end to universal primers as described previously.

TABLE 2

| | | |
|---|---|---|
| | | sequence listing |
| condon | sequence | ID no. |

forward primers

| | | |
|---|---|---|
| 420G/T | ACCAGCTTTGCCAGTTCCR | 7 |
| 416C/A | TTCCGTGGGTGTGGCX | 8 |

reverse primer

| | |
|---|---|
| GGCAGAGCGACTAAAAGCAAA | 9 |

Sequence of primers used to detect Gc1F, Gc1S and Gc2 polymorphisms. R=G or T; XC or A. 420T and 416A were attached to FAM labelled universal primer G; 420 G and 416C were attached to JOE labelled universal primer C.

TABLE 3

420

| | | G | T |
|---|---|---|---|
| 416 | A | Gc1F | Gc2 |
| | C | Gc1S | |

The Gc phenotypes are dependent upon the codon mutations detected. Note that 416A and 420T do not exist together in coupling. The 420G primer detects Gc1 phenotypes; 420T detects Gc2; 416C detects Gc2 or Gc1F (dependent on codon 420 sequence); 416A deter Gc1S.

### Explanation of Examples

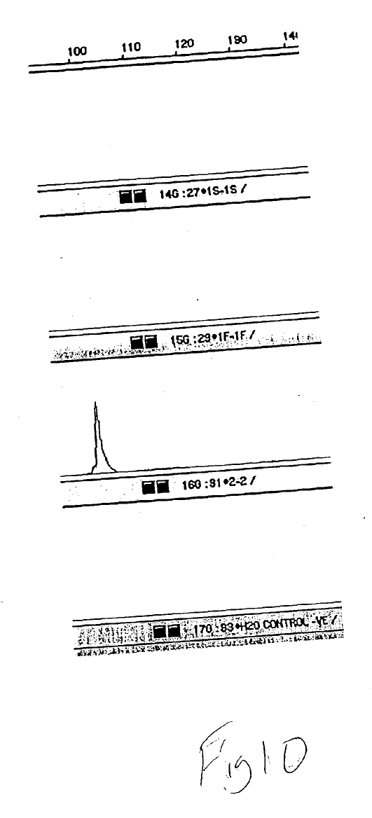

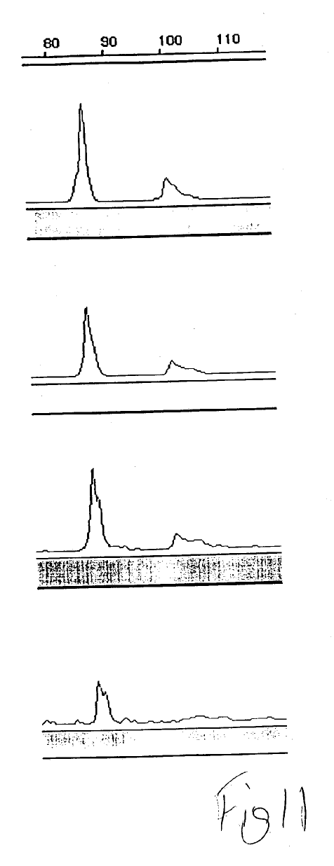

A series of examples are given (FIGS. 8 to 13). Two aspects were tested, specificity and sensitivity. To carry out specificity tests, a series of singleplex reactions were carried out. In FIG. 8, primer 416A was shown to be a specific test for the Gc1F polymorphism by testing against Gc 1S-1S; 1F-1F; 2-2 and a negative control with only the 1F-1F giving a single and no background being observed with the other samples. Similarly, primer 420G was specific for Gc1 polymorphisms (FIG. 9) with only the first two samples giving a signal; and 420T was specific for Gc2 polymorphisms (FIG. 10). The system was demonstrated to work with all primers in a cocktail mix FIGS. 11, 12 and 13). Furthermore sensitivity of detection was c. 8 pg genomic DNA (FIG. 11 and 13). A mixture was analysed (FIG. 12); this demonstrated that mixtures are easily interpreted, the different molecular weights of FAM and JOE confer different molecular weights on the DNA fragments the same size, and this facilitates interpretation. It is easy to distinguish the artefact pull-up from a true allele (FIG. 14).

Reaction Conditions

The reagent concentrations were the same as described for mitochondrial DNA. Primer concentrations used were 125 nM for the locus specific forward primers and the reverse primer. The universal forward primers were at 100 nM, and the universal reverse primer at 288 mM Locus specific and universal primers were admixed in a single tube reaction. The cycling conditions used were 94C for 30 sec; 61C for 30 sec; 72C for 90 sec for 35 cycles, followed by 72C for 10 min.

All phenotypes were verified by independent analysis using conventional isoelectric focussing.

1. A method for indicating the likelihood that a DNA mixture arose from sources of a defined type where the DNA mixture is formed by DNA samples from more than one source, the method involving:
the determination of the identity of the alleles present at a locus for the DNA in the mixture;
determining a first probability function for the situation where the DNA mixture is formed from samples arising from the given person and from a first other person;
determining a second probability function for the situation where the DNA mixture is formed from samples arising from a second other person and a first other person;
using the first probability function as numerator and the second probability function as denominator in determining a likelihood ratio for the mixture having arisen from the defined type of sources considered in the first probability function;
determining such likelihood ratios for a plurality of loci; and
combining the likelihood ratios to give a combined likelihood ratio for the mixture having arisen from the defined type of sources considered in the first probability function.

2. A method according to claim 1 in which the first probability function is the probability that the defined type provides one or both of the mixture sources based on the frequency of occurrence of the possible allele combinations which could generate the identified allele identity or identities for that locus.

3. A method according to claim 1 in which the second probability function is the probability that the first and second other persons provide the identity for the mixture sources based on the frequency of occurrence of possible allele combinations which could have generated the identified allele identity of identities for that locus.

4. A method according to claim 1 where the defined type is the given person and an unknown person, the first function is based on the frequency of occurrence of the different possible allele combinations for the unknown person which are possible knowing the given persons alleles at that locus.

5. A method according to claim 1 where the defined type is the given person and an unknown person, the second function is base on the frequency of occurrence of the different allele combinations which are possible from the two unknown persons which give the allele identity or identities obtained.

6. A method according to claim 1 where the defined type is the given person and the first other person is a known person, the first function is defined as 1.

7. A method a according to claim 1 where the defined type is the given person and the first other person is a known person, the second function is based on the frequency of occurrence of the different possible allele combinations for the unknown person which are possible knowing the known person's alleles at that locus.

8. A method according to claim 1 in which the method is applied to at least 20 loci.

9. A method according to claim 1 in which the combined likelihood ratio is obtained by multiplying the individual likelihood ratios together.

10. A method according to claim 1 in which to estimate the optimum number of loci used a theoretical likelihood ratio is used, calculated from:
$$\overline{{LR}_{n}} = {\prod }_{m = 1}^{mp} {LR}^{( fm \times n )}$$
where n is the number of loci; mp is the number of possible allele identities for a simple mixture, LR is the likelihood ratio; LR is the combined likelihood ratio; and fm is the proportion of an array of a loci having a particular mixture type m.

11. A method according to claim 1 where the allele identity or identities of a given person and/or known first other person are under consideration, the method includes the determination of the allele identity or identities at one or more of the loci under consideration from DNA obtained only from the given person or known first person.

12. A method according to claim 1 where the defined type is the given person and the first other person is a known person, such as a victim, and at least some of the loci considered in the method are those in which the given person and first other person are known to differ in allele identity.

13. A method according to claim 1 where the defined type is the given person and the first other person is a known person, such as a victim, the method considers loci at which the given person and known first person are known to have the same homozygous allele identity.

14. A method according to claim 13 in which in such cases the method includes the establishment of a probability value that the other identity is absent.

15. A method according to claim 13 in which the probability value involves an investigation of the background noise level from the allele identity investigating process and/or the introduction of one or more negative control samples and/or the determination of a cumulative probability density function for one or more or all of the negative controls.

16. A method according to claim 1 where the defined type is the given person and the fast other person is a known person, such as a victim the method involves the establishment of a probability value that the given person's allele identity or identities has not been detected.

17. A method according to claim 16 in which the probability value relates to the given person's allele identity being different from that of the known first other person's.

18. A method according to claim 16 in which the probability value relates to the given person's allele identity being the same as that of the known first other person's.

19. A method according to claim 1 in which the method further includes the prediction of the proportion of the mixture arising from the person other than the first other person, for instance from the suspect as the given person.

20. A method according to claim 1 in which the method includes an estimate or calculation of a value for p(null), the value for p(null) being calculated from a cumulative probability density function.


SEQUENCE LISTING (9 sequences)

SEQ ID NO: 1 - DNA, 20 bp, Artificial Sequence
  Description of Artificial Sequence A human mitochondrial forward primer used attached to an artificial universal primer tag to detect a SNP polymorphiusm
gtattttcgtctggggggta

SEQ ID NO: 2 - DNA, 21 bp, Artificial Sequence
  Description of Artificial Sequence A human mitochondrial forward primer used attached to an artificial universal primer tag to detect a SNP polymorphiusm
gtctgtctttgattcctgccc

SEQ ID NO: 3 - DNA, 20 bp, Artificial Sequence
  Description of Artificial Sequence A human mitochondrial forward primer used attached to an artificial universal primer tag to detect a SNP polymorphiusm
tttgattcctgcctcatccc

SEQ ID NO: 4 - DNA, 20 bp, Artificial Sequence
  Description of Artificial Sequence A human mitochondrial forward primer used attached to an artificial universal primer tag to detect a SNP polymorphiusm
atattacaggcgaacatacc

SEQ ID NO: 5 - DNA, 27 bp, Artificial Sequence
  Description of Artificial Sequence A human mitochondrial forward primer used attached to an artificial universal primer tag to detect a SNP polymorphiusm
gcttgtaggacataataataacaatta

SEQ ID NO: 6 - DNA, 22 bp, Artificial Sequence
  Description of Artificial Sequence A human mitochondrial reverse primer used attached to an artificial universal primer tag to detect a SNP polymorphism
cagagatgtgtttaagtgctgt

SEQ ID NO: 7 - DNA, 19 bp, Artificial Sequence
  Description of Artificial Sequence A human Gc forward primer used attached to an artificial universal primer tag to detect a SNP polymorphism
accagctttgccagttcck

SEQ ID NO: 8 - DNA, 21 bp, Artificial Sequence
  Description of Artificial Sequence A human Gc forward primerused attached to an artificial universal primer tag to detect a SNP polymorphism
ggcagagcgactaaaagcaaa

SEQ ID NO: 9 - DNA, 16 bp, Artificial Sequence
  Description of Artificial Sequence A human Gc reverse primer used attached to a universal primer tag to detect a SNP polymorphism
ttccgtgggtgtggcm